(19) United States
(12) Patent Application Publication
(10) Pub. No.: US 2025/0172553 A1
(43) Pub. Date: May 29, 2025

(54) INSULIN EFFICACY EVALUATION TESTER FOR AMYLOID FIBRIL

(71) Applicant: Pharmalytix, Inc., Middletown, DE (US)

(72) Inventors: Mark Steven Nissen, Evans, GA (US); Danielle Ryann Glickstein, Scotch Plains, NJ (US); Sierra Chun-Ying Houang, Woodstock, GA (US); Janie Nicole Johnson, Columbus, GA (US); Grady Ian Mosby Lee, Cataula, GA (US); Charles Gilliland, Atlanta, GA (US); Lea Gilliland, Atlanta, GA (US)

(21) Appl. No.: 18/934,653

(22) Filed: Nov. 1, 2024

Related U.S. Application Data
(60) Provisional application No. 63/595,188, filed on Nov. 1, 2023.

(51) Int. Cl. G01N 33/543; G01N 33/74

(52) U.S. Cl. CPC G01N 33/54388; G01N 33/74; G01N 2333/62

(57) ABSTRACT

An exemplary system and method are disclosed for a lateral flow immunoassay test designed for testing insulin efficacy and a method of using the device. The exemplary test, and associated device or system, specifically detects ineffective insulin by testing, in an insulin sample, for the presence of amyloid fibrils that may have formed from denatured insulin protein molecules. If amyloid fibrils are present, the antibodies conjugated with a label of the label-conjugated anti-amyloid fibrils (e.g., gold nanoparticles), of the test, would bind to the amyloid fibrils present in the sample to reveal a test line, or other indicator described herein, indicating the presence of amyloid fibrils. The test can employ a secondary antibody as a control line to detect the presence of insulin.

### CROSS REFERENCE TO RELATED APPLICATIONS

This application claims the benefit of priority to U.S. Provisional Application No. 63/595,188, filed Nov. 1, 2023, which is incorporated by reference herein in its entirety.

### BACKGROUND

Diabetic individuals rely on the administration of insulin to regulate their blood sugar. Denaturation or degradation of an insulin sample can be caused by errors in manufacturing, incorrect storage, or expiration of the insulin, among other reasons, which can reduce or completely deactivate the insulin.

Insulin is typically stored in a refrigerator at approximately, e.g., 36° F. to 46° F. Besides tracking that the insulin has been properly storage, hospitals have no way to determine its efficacy.

There is a benefit for patients, individuals, hospitals, or pharmacies to determine the quality of insulin.

### SUMMARY

An exemplary system and method are disclosed for a lateral flow immunoassay test designed for testing insulin efficacy and a method of using the device. The exemplary test, and associated device or system, specifically detects ineffective insulin by testing, in an insulin sample, for the presence of amyloid fibrils that may have formed from denatured insulin protein molecules. If amyloid fibrils are present, the antibodies conjugated with a label of the label-conjugated anti-amyloid fibrils (e.g., gold nanoparticles), of the test, would bind to the amyloid fibrils present in the sample to reveal a test line, or other indicator described herein, indicating the presence of amyloid fibrils. The test can employ a secondary antibody as a control line to detect the presence of insulin.

The lateral flow immunoassay analysis can be implemented as an at-home tester device or in benchtop laboratory equipment. An exemplary device is preferably portable and handheld to provide quick analysis while also being compatible with most insulin delivery methods and can be manufactured at a low cost.

In an aspect, a tester device (e.g., lateral flow assay) is provided comprising: a tester housing; a tester substrate located in the tester housing, the tester substrate comprising: a conjugate pad that receives an insulin sample, wherein the conjugate pad includes a label-conjugated anti-amyloid fibrils antibody; and a test pad (e.g., nitrocellulose fiber, membrane, filter) that couples to the conjugate pad to receive the label-conjugated anti-amyloid fibrils antibody, the test pad comprising a region treated with a second anti-amyloid fibrils antibody, wherein the second anti-amyloid fibrils antibody binds the amyloid fibrils, wherein the amyloid fibrils are also bound to the label-conjugated anti-amyloid fibrils antibody, thereby generating a visual indication of presence of amyloid fibril in the insulin sample (e.g., as an indication of insulin spoilage or degradation).

In some embodiments, the label of the label-conjugated anti-amyloid fibrils antibody is gold nanoparticles, colloidal gold, silver nanoparticles, platinum nanoparticles, silica nanoparticles, latex (polystyrene) spheres, cellulose nanobeads, quantum dots, fluorescent quenching material, up-converting nanoparticles, fluorescent microspheres, fluorochrome dyes, fluorescent europium, fluorescent beads, lanthanide, carbon nanotubes, colloidal carbon, magnetic particles, enzymes, liposomes, fluorescent dyes, biotin, luminescent particles, or any combinations thereof. In some embodiments, a single label-conjugated anti-amyloid fibrils antibody is employed. In other embodiments, multiple of label-conjugated anti-amyloid fibrils antibody may be concurrently employed.

In some embodiments, the nanoparticle-conjugated anti-amyloid fibrils antibody has a concentration of from about 1 mg/mL to about 4 mg/mL.

In some embodiments, the test pad includes an indication line or stripe, wherein said indication line or stripe comprises the second anti-amyloid fibrils antibody.

In some embodiments, the test pad includes a second indication line or stripe as a control indicator configured to bind to unbound conjugated antibodies.

In some embodiments, the conjugate pad is coated with a label buffer solution for label-conjugated anti-amyloid fibrils antibody release.

In some embodiments, the tester input comprises a label buffer solution for label-conjugated anti-amyloid fibrils antibody release.

In some embodiments, the received insulin sample was treated with a label buffer solution for label-conjugated anti-amyloid fibrils antibody release.

In some embodiments, the tester device further comprises a sample pad as the tester input, the sample pad configured to receive the insulin sample from a syringe, pen, pump, or patch.

In some embodiments, the tester device further comprises the sample pad as the tester input, the sample pad configured to contact and extract the insulin sample from an insulin pen, an insulin patch, an insulin pump, or an insulin vial.

In some embodiments, the tester device further comprises a wicking pad that couples to the test pad.

In some embodiments, the label can be detected when the insulin sample comprises at least 10% amyloid fibrils.

In another embodiment, a test kit is provided comprising: a tester comprising: a tester housing; a tester substrate located in the tester housing, the tester substrate comprising: a conjugate pad that receives an insulin sample, wherein the conjugate pad includes a label-conjugated anti-amyloid fibrils antibody; and a test pad (e.g., nitrocellulose fiber, membrane, filter) that couples to the conjugate pad to receive the label-conjugated anti-amyloid fibrils antibody, the test pad comprising a region treated with a second anti-amyloid fibrils antibody, wherein the second anti-amyloid fibrils antibody binds the amyloid fibrils, wherein the amyloid fibrils are also bound to the label-conjugated anti-amyloid fibrils antibody, thereby generating a visual indication of presence of amyloid fibril in the insulin sample (e.g., as an indication of insulin spoilage or degradation).

In some embodiments, the conjugate pad includes gold nanoparticle-conjugated anti-amyloid fibrils antibody.

In some embodiments, the nanoparticle-conjugated anti-amyloid fibrils antibody has a concentration of from about 1 mg/mL to about 4 mg/mL.

In some embodiments, the test pad includes (i) an indication line or stripe of anti-amyloid fibrils antibody configured to bind to amyloid fibrils-conjugated antibody complexes and (ii) a second indication line or stripe as the control line configured to bind to unbound conjugated antibody.

In some embodiments, the conjugate pad or the tester input comprises a buffer for nanoparticle-conjugated anti-amyloid fibrils antibody release.

In some embodiments, the test kit further comprises: a buffer for nanoparticle-conjugated anti-amyloid fibrils antibody release.

In some embodiments, the tester device includes a sample pad as the tester input, the sample pad configured (i) to receive the insulin sample (e.g., from a syringe, pen, pump, vial, etc.) (ii) to contact and extract the insulin sample (e.g., from a syringe, pen, pump, vial, etc.) or (iii) contact and extract the insulin sample (e.g., from a syringe, pen, pump, vial, etc.).

In another aspect, a method is provided comprising: providing a portable tester device comprising (i) a conjugate pad that includes label-conjugated anti-amyloid fibrils antibody and (ii) a test pad (e.g., nitrocellulose fiber, membrane, filter) treated with a second anti-amyloid fibrils antibody, wherein the second anti-amyloid fibrils antibody binds the amyloid fibrils, wherein the amyloid fibrils are also bound to the label-conjugated anti-amyloid fibrils antibody, thereby generating a visual indication of the presence of amyloid fibril in the insulin sample; receiving an insulin sample at an input of the portable tester device; activating the label-conjugated anti-amyloid fibrils antibody with a label buffer solution; generating a visual indication of presence of amyloid fibril or denatured insulin protein in a conjugated insulin sample as an indication of insulin spoilage or degradation.

In some embodiments, the method further comprises, before receiving the insulin sample at the input of the portable tester device, treating the insulin sample with a label buffer solution for label-conjugated anti-amyloid fibrils antibody release.

In some embodiments, the visual indication is generated at the portable tester device or at a device interrogating the portable tester device.

In some embodiments, the label can be detected when the insulin sample comprises at least 10% amyloid fibrils.

In another aspect, a method of treating a subject in need of insulin is provided, the method comprising: determining whether an insulin sample is safe for use by determining whether the sample has a pre-defined exceeded threshold amount of amyloid fibrils (e.g., a significant amount of amyloid fibrils), wherein presence of exceeded threshold amount of amyloid fibrils indicates the insulin sample is not safe for use.

In another aspect, a method of treating a subject in need of insulin is provided, the method comprising: determining whether the insulin sample has amyloid fibrils, wherein if it does, the subject is treated with a different insulin sample, and if it doesn't, the subject is treated with the insulin sample which has been measured.

In some embodiments, the determining is based on the insulin sample comprising at least 10% amyloid fibrils.

Other systems, methods, features, and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

The skilled person in the art will understand that the drawings described below are for illustration purposes only.

FIGS. 1A and 1B each depicts an exemplary lateral flow immunoassay test for evaluating insulin efficacy in accordance with an illustrative embodiment.

FIGS. 2A and 2B show a method of operation for the device of FIG. 1 in accordance with an illustrative embodiment.

FIGS. 3A-3E shows an example housing for the device of FIG. 1A in accordance with an illustrative embodiment.

FIG. 4 shows an example fabricated device.

### DETAILED DESCRIPTION

It is appreciated that certain features of the disclosure, which are, for clarity, described in the context of separate embodiments, can also be provided in combination with a single embodiment. Conversely, various features of the disclosure, which are, for brevity, described in the context of a single embodiment, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Example Test

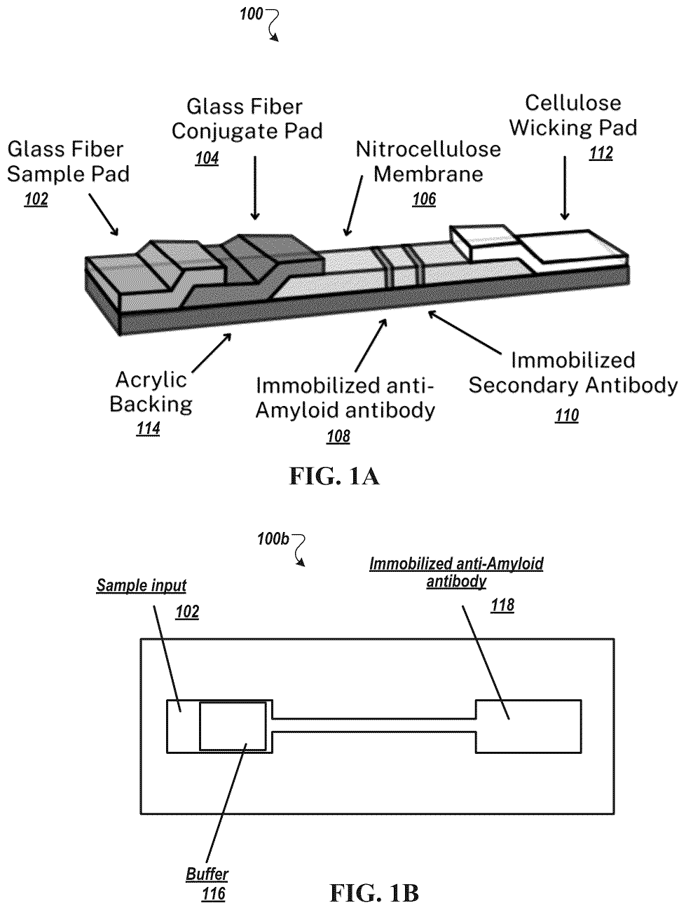

FIGS. 1A and 1B each shows an exemplary device 100 for a lateral flow immunoassay test designed for testing insulin efficacy in accordance with an illustrative embodiment. The device 100, as a portable tester in the example of FIG. 1A, includes a sample input 102 (shown as “glass fiber sample pad” 102), a conjugate pad 104 (shown as “glass fiber conjugate pad” 104), test pad 106 (shown as “nitrocellulose membrane” test pad 106, and a wicking pad 112 (show as “cellulose wicking pad” 112) formed on a substrate 114 (shown as “acrylic backing” 114). The substrate 114 can be alternatively made of acrylonitrile styrene acrylate (ASA), polypropylene, polyethylene, acrylic, polyoxymethylene, polyethylene terephthalate, polycarbonate, polyvinyl chloride, or combinations thereof. FIG. 1B shows a device 100 (shown as 100b) configured for benchtop analysis having the sample input 102, a buffer region 116 (shown as “buffer” 116), and test region 118 (shown as “Immobilized anti-Amyloid antibody). The device 100b may be configured as a microfluidic or fluidic device for testing insulin in combination with an instrument (not shown). Example instruments may include colorimetric or electrochemical detectors.

Referring to FIG. 1A, the sample pad 102 serves as an input for an insulin sample. The input can have an assembly to couple to, or contact parts of, a tip of a syringe, a tip of an inulin pen, a connector or disposable infusion adapter, e.g., cannula, of an insulin pump, or patch having insulin. In some embodiments, the sample pad 102 is fabricated of a glass fiber to contact and extract an insulin sample from an insulin pen, an insulin patch, an insulin pump, or an insulin vial. In some embodiments, the glass fiber sample pad 102 includes a label buffer solution for label-conjugated anti-amyloid fibrils antibody release. In other embodiment, the received insulin sample is treated with the label buffer solution for label-conjugated anti-amyloid fibrils antibody release. An example label buffer solution may include Tris-HCl, Triton X-100, Tween 20, phosphate buffer, phosphate buffered saline, or combinations thereof.

The conjugate pad 104 includes a label-conjugated anti-amyloid fibrils antibody and is coated with a label buffer solution for label-conjugated anti-amyloid fibrils antibody release. In the example shown in FIG. 1, the conjugate pad 104 is made of glass fiber and is coupled to the sample pad with a portion located beneath the sample pad 102 to receive the insulin sample from the glass fiber sample pad 102. In some embodiments, the

In some embodiments, the conjugate pad 104 includes gold nanoparticles for the label. In alternative embodiments, the label(s) can be colloidal gold, silver nanoparticles, platinum nanoparticles, silica nanoparticles, latex (polystyrene) spheres, cellulose nanobeads, quantum dots, fluorescent quenching material, up-converting nanoparticles, fluorescent microspheres, fluorochrome dyes, fluorescent europium, fluorescent beads, lanthanide, carbon nanotubes, colloidal carbon, magnetic particles, enzymes, liposomes, fluorescent dyes, biotin, luminescent particles, or any combinations thereof.

In some embodiments, the nanoparticle-conjugated anti-amyloid fibrils antibody has a concentration of from about 1 mg/mL to about 4 mg/mL, for example, about 1.25 mg/mL, about 1.5 mg/mL, about 1.75 mg/mL, about 2 mg/mL, about 2.25 mg/mL, about 2.5 mg/mL, about 2.75 mg/mL, about 3 mg/mL, about 3.25 mg/mL, and about 3.75 mg/mL.

The test pad 106 includes regions of immobilized antibodies. In some embodiments, the test pad 106 is made of nitrocellulose membrane and is coupled to the conjugate pad 104 to receive the label-conjugated anti-amyloid fibrils antibody. In other embodiments, the test pad 106 is made from nitrocellulose fiber or a nitrocellulose filter.

In the example shown in FIG. 1A, the test pad 106 includes two regions of immobilized antibodies in which the first region is an indication line 108 (also referred to as “immobilized anti-amyloid antibody” or “test line”) treated with an anti-amyloid fibrils antibody that can bind the amyloid fibrils. The amyloid fibrils are also bound to the label-conjugated anti-amyloid fibrils antibody, thereby generating a visual indication of the presence of amyloid fibril in the insulin sample (e.g., as an indication of insulin spoilage or degradation). The label can be detected when the insulin sample comprises at least 10% amyloid fibrils, or at least 9% amyloid fibrils, or at least 8% amyloid fibrils, or at least 7% amyloid fibrils, or at least 6% amyloid fibrils, or at least 5% amyloid fibrils. The second region, in the example, is a control line 110 (also referred to as “immobilized secondary antibody”) configured to bind to unbound conjugated antibody.

The wicking pad 112 is configured to draw the sample through the test pad. In the example shown in FIG. 1A, the wicking pad 112 is made of cellulose and is coupled to the test pad 106.

### Example Method of Operation

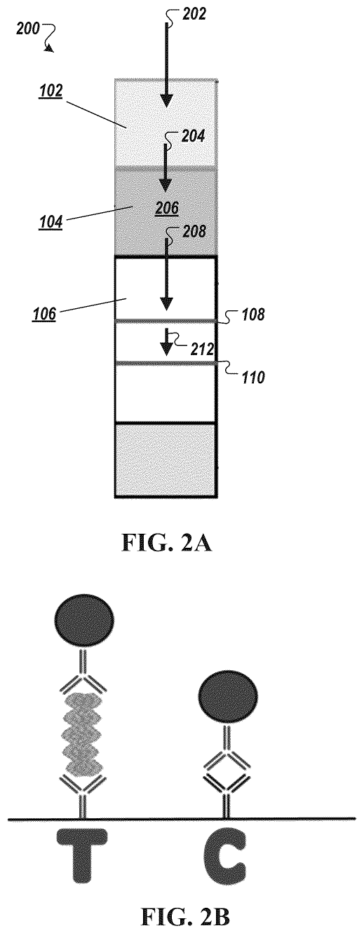

FIG. 2A shows an example method 200 of operation for the device 100 described in relation to FIG. 1A. In FIG. 2A, the portable tester device 100 is configured to detect the presence of amyloid fibrils, primarily those present in degraded insulin (e.g., heat degraded insulin). A sample of insulin is applied (202) to the sample pad 102 of the test device, and the antibodies (108, 110) can provide a visual confirmation of the presence of amyloid fibrils.

During the operation of the portable tester device 100, the user can place (202) a sample (e.g., 100 uL sample) of insulin via a syringe or pen onto the sample pad 102. The sample flows (204) to the conjugate pad (104), which, in this example, includes a buffer comprising label-conjugated anti-amyloid fibrils antibody that can mix with the sample. If the sample has heat-degraded amyloid fibrils, it will bind (206) to the label-conjugated anti-amyloid fibrils antibody in the conjugate pad. The exemplary device is an iteration on a “sandwich” immunoassay. The total solution, containing a bound sample and unbound conjugated antibody, is activated with a label buffer solution and can then flow (208) through the test pad 106. The test pad 106 includes an indication line 108 treated with an anti-amyloid fibrils antibody that can bind the amyloid fibrils. There, any amyloid fibrils-conjugated antibody complexes will first bind 210 to the antibody in the indication line. This creates the sandwich, as shown in FIG. 2B of immobilized antibody, amyloid fibril, mobile antibody, label (shown as test line “T”). The control line “C” does not include the antibody.

If sufficient sample-antibody complexes bind 210 at the test line 108, the label will become detectable (e.g., visually) as an indication of insulin spoilage or degradation. In some embodiments, a device may be employed to enhance the visualization of the indicator, e.g., UV light, a colored light, or scintillator. In some embodiments, the test line may include an optical component to magnify the indication.

The label can be configured to provide detection when the insulin sample comprises at least 10% amyloid fibrils, or at least 9% amyloid fibrils, or at least 8% amyloid fibrils, or at least 7% amyloid fibrils, or at least 6% amyloid fibrils, or at least 5% amyloid fibrils.

Referring still to FIG. 2A, The remaining unbound conjugated antibody would then flow (212) to the control line 110 having a control antibody (e.g., goat-anti-rabbit). At the control line, in this example, the anti-amyloid fibrils antibody would bind 514 to the control antibody 110, creating another sandwich: immobilized control antibody, anti-amyloid fibrils, and labels. If the test had worked correctly, the control line 110 would be visible independent of the visibility of the test line 108.

Because the label buffer has been applied and dried during manufacturing in the conjugate pad 104, there are no additional steps for the user: no mixing reagents, no extra components, and no confusing instructions. All the end user needs to do is drop the insulin sample onto the glass fiber sample pad and set a timer (e.g., for 30 minutes.) This allows the device to be used in pharmacies and clinics, and at home.

The device can be used for treating a subject in need of insulin. In an embodiment, the subject can be treated by determining whether an insulin sample is safe for use by determining whether the sample has a pre-defined exceeded threshold amount of amyloid fibrils (e.g., a significant amount of amyloid fibrils), wherein the presence of an exceeded threshold amount of amyloid fibrils indicates the insulin sample is not safe for use. In another embodiment, the subject can be treated by determining whether the insulin sample has amyloid fibrils, wherein if it does, the subject is treated with a different insulin sample, and if it doesn't, the subject is treated with the insulin sample that has been measured. In some embodiments, the determining is based on the insulin sample comprising at least 10% amyloid fibrils, or at least 9% amyloid fibrils, or at least 8% amyloid fibrils, or at least 7% amyloid fibrils, or at least 6% amyloid fibrils, or at least 5% amyloid fibrils.

### Example Configuration

Table 1 shows an exemplary configuration for the test device and its method of use.

TABLE 1

1. Take a few drops of insulin from a vial (around 100 μL).
2. Place the insulin on the glass fiber membrane sample pad.
3. The absorbent pad, made from cellulose fibers, on the opposite side
of the test will wick the insulin through the test.
4. After the insulin passes through the glass fiber membrane, it will go
into another glass fiber membrane, which has been soaked in a running
buffer and dried then soaked in a gold nanoparticle conjugated-
antibody solution and dried. These antibodies are anti-amyloid fibrils.
Amyloid fibrils are a structure that forms during heat degradation of
insulin.
5. As the insulin passes through the treated pad, amyloid fibrils in heat-
degraded insulin will bind to the antibodies. The wicking will pull the
bound insulin, the unbound insulin, and the unbound conjugated
antibodies through the test.
6. As the solution passes through to the nitrocellulose layer, it will pass
by immobilized anti-amyloid fibril antibodies, where the bound insulin
will bind again. This will produce the red test line.
7. Further down the nitrocellulose membrane, the solution will pass by
immobilized secondary antibodies. Any antibodies that did not bind
at the test line will bind to these antibodies and form the red
control line.

The flow of insulin could take anywhere from 15-30 minutes, depending on the viscosity of the insulin, which can change based on the level of degradation.

### Example Device Casing

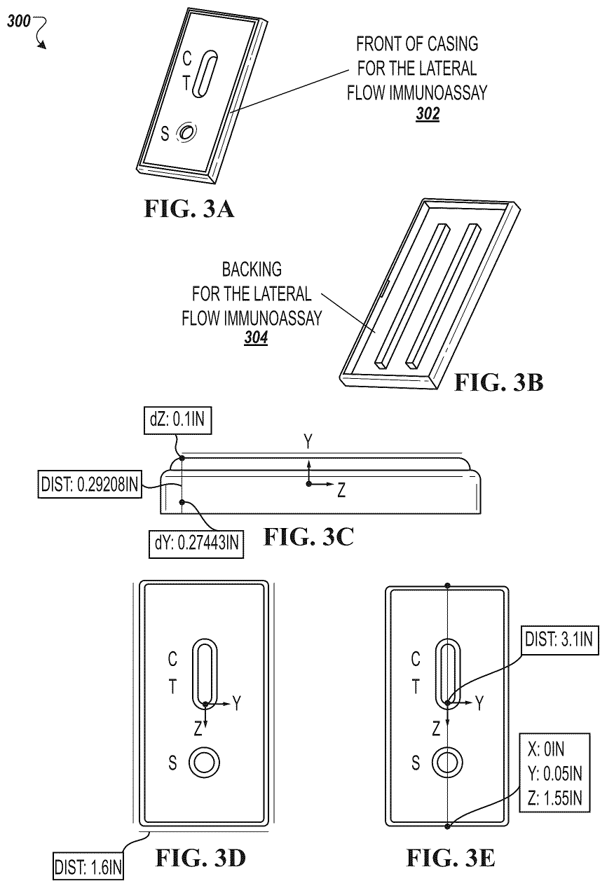

FIGS. 3A-3E show an exemplary tester housing 300 for encompassing the exemplary tester substrate 100. The tester housing 300 includes a front casing 302 and a back casing 304. The tester housing may be made of acrylonitrile butadiene styrene (ABS), acrylonitrile styrene acrylate (ASA), polypropylene, acrylic, polystyrene, polyester, polyethylene, polycarbonate, or combinations thereof. FIGS. 3C-3E shows exemplary dimensions for the exemplary tester housing 300. FIG. 3C shows an assembled view of the device.

In some embodiments, the exemplary device can be supplied as a test kit. In some embodiments, the test kit can further include a buffer for nanoparticle-conjugated anti-amyloid fibrils antibody release. In some embodiments, the buffer comprises Tris-HCl, Triton X-100, Tween 20, phosphate buffer, phosphate buffered saline, or combinations thereof.

### Experimental Result and Additional Examples

A study was conducted to develop and test an exemplary device.

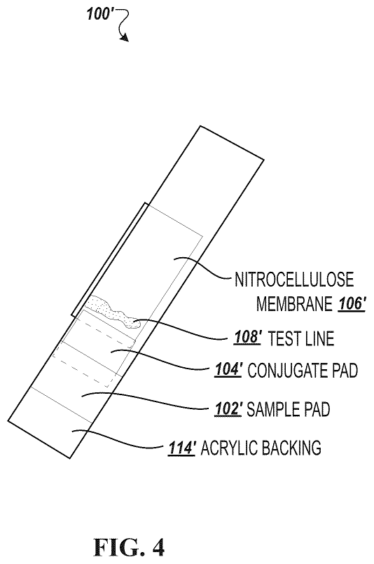

FIG. 4 shows an image of a fabricated device used in the study. The device was evaluated to test the efficacy of insulin. The immunoassay was conducted on a clear acrylic backing. A sample of insulin was applied to the sample pad, and the insulin began to flow laterally across the assay. After traveling across the sample pad, the insulin reached the conjugate pad, which was soaked in gold nanoparticles. Since the insulin used in the study had been partially denatured with heat, it had formed amyloid fibrils to which the gold nanoparticles on the device bound. After traveling through the conjugate pad, the sample reached the nitrocellulose pad, where the antibody was located. The antibody was hand-piped onto the nitrocellulose membrane to form the test line. Due to the imprecise nature of hand piping the antibody onto the nitrocellulose membrane, the line was not as rigid as it could be with more precise manufacturing methods. The sample of insulin with the amyloid fibrils flowed down the assay until it reached the test line, where the amyloid fibrils were then bound to the anti-amyloid antibodies. Since the amyloid fibrils had already bonded with the gold nanoparticles, a visible line was formed at the test line which can be seen in FIG. 4.

It was observed that a portion of the insulin which had not been denatured flowed further down the membrane past the test line but was not bound to gold nanoparticles. This was a positive result, as the gold nanoparticles were only attached to the amyloid fibrils, and the amyloid fibrils primarily attached to the antibodies, yielding a visible positive test line. This study was conducted to demonstrate the test because it was not easily viewed within the 3D-printed housing for the test.

Fabrication. Prior to assembly of the device, the gold nanoparticles were first conjugated to the anti-amyloid fibrils antibody. Gold nanoparticles were obtained from Cytodiagnostics, and anti-amyloid fibrils were obtained from Millipore Sigma. The conjugation process begins with filter purification of the raw antibody. 80 μL of the antibody were centrifuged at 14,000×G for 10 minutes in a 150 kDa filter (Millipore). The concentrated antibody was then resuspended in the conjugation buffer provided by Cytodiagnostics, and concentration was determined using a BCA Assay (ThermoFisher) and plate reader (ThermoFisher). The next procedure is a pH and concentration optimization using a pH range of 7.8 to 9.8 and a concentration range of 0.1 to 4 mg/mL. The optimal solution is determined using Equation 1.

$$ratio = ( \frac{Absorbance @ 690 nm sample}{Absorbance @ 530 nm sample} ) - ( \frac{Absorbance @ 690 nm control}{Absorbance @ 530 nm control} ) \qquad ( Eq . 1 )$$

Obtaining a ratio close to zero is desirable. The full volume of antibody is conjugated to the gold nanoparticles at the appropriate concentration and pH overnight. In preparation for assembly, the conjugate pad is first coated with a buffer to improve gold nanoparticle release [24]. After drying, the conjugate pad is then dipped in the conjugated antibody solution and then dried again. The immobilized anti-amyloid fibrils and goat-anti-rabbit antibodies are striped onto the nitrocellulose membrane and dried. Assembly consists of an acrylic backing card, layered with the striped nitrocellulose membrane, cellulose wicking pad, conjugate pad, and sample pad.

The study considered several design considerations, including portability and fast results. Table 2 shows example design considerations employed in the study.

TABLE 2

| Input | User Need | Design Input | Metric |
|---|---|---|---|
| Performance | Fast Results | The results should come | <30 minutes |
| | | back faster than if they | |
| | | waited to see glucose levels | |
| | | drop | |
| | Sensitivity | Must be comparable to | ≥83.5% True |
| | | current Diabetic diagnostic | positive rate |
| | | tests in sensitivity | |
| | Specificity | Must be comparable to | ≥95% True |
| | | current Diabetic diagnostic | Negative rate |
| | | tests in specificity | |
| | Detection Range | Must be able to detect | >0.1% amyloid |
| | | presence of amyloid fibrils | fibril concentration |
| | | | by volume |
| Physical | Portability | Users must be able to bring | ≤144 in³ |
| Characteristics | | home and travel with device | |
| | Cost of Materials | Device has to be affordable | <$36 per test |
| | | to users | |
| | Compatibility | Must be able to be used with | Able to be used |
| | | vials and insulin pens | with ≥75% of |
| | | | insulin delivery |
| | | | systems |
| | No Contamination | Device must not | ≤.3 μL new material |
| | | contaminate insulin | after use |
| User Interface | Ease of Use | Test results must be easy to | ≥85% acceptance |
| | | read and understand | rate in usability |
| | | | study |
| | Instructions | Instructions must be | ≥85% acceptance |
| | | comprehensive and easy to | rate in usability |
| | | follow | study |
| Reliability and | Usable Shelf Life | Any single-use components | ≥3 months after |
| Maintenance | | must be shelf-stable for 3 | opening |
| | | months after opening | |
| | | Any single-use components | ≥8 months before |
| | | must be shelf-stable for 8 | opening |
| | | months before opening | |
| Packaging and | Easy to understand | Principal display panel | ≤6″ × 8″ |
| Labeling | and use as an over- | should be large enough to | |
| | the-counter product | accommodate all mandatory | |
| | | labeling information | |
| | | Principal display panel must | “Rapid Insulin |
| | | contain statement of identity | Efficacy Test” or |
| | | | equivalent |
| | | All display panels must have | N/A |
| | | a declaration of net quantity | |
| | | of contents in appropriate | |
| | | units | |
| | Safe | Packaging must be made of | No known or |
| | | known and traceable | traceable materials |
| | | materials | |
| | | Packaging materials must | Non-toxic, non- |
| | | not be harmful | leaching, odorless |
| | | Packaging must allow | N/A |
| | | sterilization | |
| | | Packaging must maintain | ≥8 months |
| | | sterility up to use | |

In Table 2, portability was defined as less than 144 in³. To assess the metric, the prototype was measured in SolidWorks, which provided a measure of 0.274 inches×1.6 inches×3.1 inches to provide a volume of 1.35 inches³.

To evaluate the time, a quantitative model described by Berli and Kler was used²⁵. The reaction volume (V_{L}) was calculated using Equation 2.

$${V}_{L} = {w}_{L} {w}_{s} h \qquad ( Eq . 2 )$$

In Equation 2, w_{L} refers to the test line width, wₛ is the width of the strip, and h is the height of the assay paper. Since the exemplary device uses both a test and control line, the reaction volume includes both of these lines as well as the space in between the two areas.

The rate of analyte capture (r) was determined using Equation 3.

$$r = {k}_{b} {C}_{A} {C}_{S} - {k}_{u} {C}_{AS} \qquad ( Eq . 3 )$$

In Equation 3, k_{b} and kᵤ are the binding and unbinding capacity of the components, C_{A} is the concentration of the analyte, C_{S} is the concentration of the antibody, and C_{AS} is the concentration of the complex formed. There are three main reactions happening in the device: binding of the amyloid to the mobile gold-conjugated anti-amyloid antibody on the conjugate pad, binding of the amyloid-antibody complex to the immobile anti-amyloid antibody on the test line, and binding of the mobile anti-amyloid antibody to the immobile secondary antibody on the control line. As such, all three reactions had to be accounted for when calculating the estimated results time.

The dynamics of capillary imbibition were also taken into account by calculating both the average velocity of the fluid front and the velocity at the sample detection zone. The average velocity of the fluid front was calculated using Equation 4.

$$u ( x ) = c / x \qquad ( Eq . 4 )$$

In Equation 4, c is a coefficient dependent on media and medium, and x is distance along the assay. For water in nitrocellulose, c is roughly equal to 1-10 mm²/s. The lower end of this range was used for these calculations with the assumption that the solution has similar properties to that of water.

Using these dynamics, rates, and corresponding parameters, three characteristic times were calculated. These characteristic times include residence time—the time the analyte spends in the capture zones, diffusive time—the time the solution takes to diffuse to the end of the assay, and characteristic kinetic time—the time taken for the reactions to occur. These calculations gave a final estimated results time of 26.54 minutes. In the final prototype, the results appeared in the 22nd minute. This time does not account for the appearance of the control line, as the secondary antibody was stored improperly and denatured, but it is estimated that this reaction would not add much time. Therefore, the design meets the critical design input of less than 30 minutes.

### Discussion

More than 1 in 10 Americans has diabetes, and 1.9 million of them have type I diabetes [1,2]. All type I diabetics and some type II diabetics depend on insulin to regulate their blood sugar [1]. Occasionally, a diabetic's blood sugar will not respond after administering insulin. Without a reliable way to determine at what point in insulin administration an issue occurred, users have to troubleshoot the entire process. During this, the user would be feeling the symptoms of hyperglycemia: headaches, nausea, vomiting, and potentially diabetic ketoacidosis. Additionally, the user has the potential to throw away hundreds of dollars in insulin and pump supplies before knowing what is wrong. Amyloid fibrils develop when insulin degrades, typically due to storage outside the acceptable temperature range. This can cause a nonresponse after administration, however, the presence of amyloid fibrils cannot be determined visually.

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various embodiments, elements, and features of the disclosed invention also include the more limited embodiments of “consisting essentially of” and “consisting of.”

As used herein, the singular forms “a,” “an,” and “the” include plural referents unless the context clearly dictates otherwise. Thus, for example, reference to a “polymer” includes embodiments having two or more such polymers unless the context clearly indicates otherwise.

Ranges can be expressed herein as from “about” one particular value and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

The following patents, applications, and publications as listed below and throughout this document are hereby incorporated by reference in their entirety herein.

### REFERENCE LIST

[1] The Facts, Stats, and Impacts of Diabetes. Centers for Disease Control and Prevention. https://www.cdc.gov/diabetes/library/spotlights/diabetes-facts-stats.html#:˜:text=37.3%20million%20Americans% E2%80%94about%201,t%20know%20they%20have%20it. Published Jan. 24, 2022. Accessed Dec. 11, 2022.
[2] Type 1 diabetes statistics. Beyond Type 1. https://beyondtype1.org/type-1-diabetes-statistics/. Published Sep. 19, 2022. Accessed Sep. 26, 2022.
[3] Mori W, Kaneko N, Nakanishi A, Zako T, Masumoto J. Insulin amyloid fibrils interact directly with the NLRP3, resulting in inflammasome activation and pyroptotic cell death. International journal of immunopathology and pharmacology. https://www.ncbi.nlm.nih.gov/pmc/articles/PMC8371720/. Published Aug. 16, 2021. Accessed Nov. 1, 2022.
[4] Mordor Intelligence. DIABETES CARE DRUGS MARKET-GROWTH, TRENDS, COVID-19 IMPACT, AND FORECASTS (2022-2027). Accessed Dec. 12, 2022. https://www.mordorintelligence.com/industry-reports/diabetes-drugs-market#:˜:text-Based%20on%20drugs%2C%20the%20insulin%20segment%20holds%20a,10% 25%20and%2025%25%20of%20people%20with%20Type-2%20diabetes
[5] Hayes, T, Farmer J. Insulin Cost and Pricing Trends. American Action Forum. Apr. 2, 2020. Accessed Dec. 12, 2022. https://www.americanactionforum.org/research/insulin-cost-and-pricing-trends/[6]
[6] Liberato, Eric, Oct. 7, 2022, Sierra Houang & Danielle Glickstein, Discussion of why insulin and other biologics don't currently use temperature indicators.
[7] Dr. Gilliand, Charles & Dr. Gilliland, Lea & Gilliland, Mary Catherine, Aug. 28, 2022, Danielle Glickstein & Sierra Houang & Janie Johnson & Grady Lee & Mark Nissen, Discussion of problem and team introductions.
[8] Dr. Thoman, Nina and Smith, Courtney, Sep. 7, 2022, Sierra Houang & Grady Lee, Discussion of storage and handling of insulin for diabetic students at Georgia Tech.
[9] Cornell, Abby, Sep. 15, 2022, Sierra Houang & Janie Johnson, Interview about experiences as a type I diabetic.
[10] Dr. Davis, Georgia, Sep. 27, 2022, Janie Johnson & Grady Lee, Discussion of type 1 diabetic patients and experience of patients of varying socioeconomic statuses.
[11] Spalding, Art & Koushik, Shree, Oct. 14, 2022, Sierra Houang & Grady Lee, Discussion of FDA medical device regulations and device patentability.
[12] Surmacz-Chwedoruk W, Malka I, Bożycki Ł, Nieznańska H, Dzwolak W. On the heat stability of amyloid-based biological activity: Insights from thermal degradation of insulin fibrils. PLOS ONE. https://journals.plos.org/plosone/article?id=10.1371%2Fjournal.pone.0086320. Published Jan. 21, 2014. Accessed Nov. 1, 2022.
[13] Dumlao J. Type 2 diabetes: How long does it take for insulin to work? Healthline. https://www.healthline.com/health/type-2diabetes/basal-insulin/how-long-for-insulin-to-work. Published Sep. 20, 2022. Accessed Sep. 26, 2022.
[14] Nayeri F, Shariat M, Mousavi Behbahani H M, Dehghan P, Ebrahim B. Blood glucose measurement by glucometer in comparison with standard method in diagnosis of neonatal hypoglycemia. Acta Med Iran. 2014; 52 (8): 619-622.
[15] Amazon.com: DSS Precision Xtra Blood Glucose Meter Kit, results in 5 . . . https://www.amazon.com/DSS-Precision-Glucose-Results-Included/dp/B00JIK6CIW. Accessed Sep. 26, 2022.
[16] FreeStyle InsuLinx Test Strips. Diabetic Warehouse. 2022. Accessed 19 Sep. 2022.
[17] Kesavadev J, Saboo B, Krishna M B, Krishnan G. Evolution of Insulin Delivery Devices: From Syringes, Pens, and Pumps to DIY Artificial Pancreas. Diabetes Ther. 2020 June; 11 (6): 1251-1269. doi: 10.1007/s13300-020-00831-z. Epub 2020 May 14. PMID: 32410184; PMCID: PMC7261311.
[18] Herdman M L, Larck C, Schliesser S H, Jelic T M. Biological contamination of insulin pens in a hospital setting. Am J Health Syst Pharm. 2013; 70 (14): 1244-1248. doi: 10.2146/ajhp120728.
[19] Home. https://kebs.isolutions.iso.org/obp/ui#!iso:std:iso:9241:-11:ed-2:v1:en. Accessed Sep. 26, 2022.
[20] How to store blood glucose test strips. How to store test strips. https://www.bionime.com/msg/how-to-store-strips-properly.html#:˜:text=Test%20strips%20are%20effective%20for,prevent%20the%20ingress%20of%20humidity. Accessed Sep. 26, 2022.
[21] Zolotukhina E V, Gerasimova E V, Sorokin V V, Levchenko M G, Freiman A S, Silina Y E. The Impact of the Functional Layer Composition of Glucose Test-Strips on the Stability of Electrochemical Response. Chemosensors. 2022; 10 (8): 298. https://doi.org/10.3390/chemosensors10080298
[22] 21CFR801 Labeling for in vitro diagnostic products. Code of Federal Regulations. https://www.ecfr.gov/current/title-21/chapter-I/subchapter-H/part-809/subpart-B/section-809.10. Accessed Sep. 26, 2022.
[23] 809.10 Labeling for in vitro diagnostic products. Code of Federal Regulations. https://www.ecfr.gov/current/title-21/chapter-I/subchapter-H/part-809/subpart-B/section-809.10. Accessed Sep. 26, 2022.
[24] Srisrattakarn A, Tippayawat P, Chanawong A, et al. Development of a Prototype Lateral Flow Immunoassay for Rapid Detection of Staphylococcal Protein A in Positive Blood Culture Samples. Diagnostics (Basel). 2020; 10 (10): 794. Published 2020 Oct. 7. doi: 10.3390/diagnostics10100794
[25] Berli C L, Kler P A. A quantitative model for lateral flow assays. Microfluidics and Nanofluidics. 2016; 20 (7). doi: 10.1007/s10404-016-1771-9.
[26] U.S. Pat. No. 7,932,099
[27] U.S. Pat. No. 7,459,314

What is claimed is:

1. A tester device comprising:
a tester housing;
a tester substrate located in the tester housing, the tester substrate comprising:
a conjugate pad that receives an insulin sample, wherein the conjugate pad includes a label-conjugated anti-amyloid fibrils antibody; and
a test pad that couples to the conjugate pad to receive the label-conjugated anti-amyloid fibrils antibody, the test pad comprising a region treated with a second anti-amyloid fibrils antibody, wherein the second anti-amyloid fibrils antibody binds the amyloid fibrils, wherein the amyloid fibrils are also bound to the label-conjugated anti-amyloid fibrils antibody, thereby generating a visual indication of presence of amyloid fibril in the insulin sample.

2. The tester device of claim 1, wherein the label of the label-conjugated anti-amyloid fibrils antibody is gold nanoparticles, colloidal gold, silver nanoparticles, platinum nanoparticles, silica nanoparticles, latex (polystyrene) spheres, cellulose nanobeads, quantum dots, fluorescent quenching material, up-converting nanoparticles, fluorescent microspheres, fluorochrome dyes, fluorescent europium, fluorescent beads, lanthanide, carbon nanotubes, colloidal carbon, magnetic particles, enzymes, liposomes, fluorescent dyes, biotin, luminescent particles, or any combinations thereof.

3. The tester device of claim 1, wherein a single label-conjugated anti-amyloid fibrils antibody is employed.

4. The tester device of claim 1, wherein multiple of label-conjugated anti-amyloid fibrils antibody may be concurrently employed.

5. The tester device of claim 1, wherein the nanoparticle-conjugated anti-amyloid fibrils antibody has a concentration of from about 1 mg/mL to about 4 mg/mL.

6. The tester device of claim 1, wherein the test pad includes an indication line or stripe, wherein said indication line or stripe comprises the second anti-amyloid fibrils antibody.

7. The tester device of claim 6, wherein the test pad includes a second indication line or stripe as a control indicator configured to bind to unbound conjugated antibodies.

8. The tester device of claim 1, wherein:
(i) the conjugate pad is coated with a label buffer solution for label-conjugated anti-amyloid fibrils antibody release; and/or
(ii) the tester input comprises a label buffer solution for label-conjugated anti-amyloid fibrils antibody release.

9. The tester device of claim 1, further comprising a sample pad as the tester input, the sample pad configured to:
(i) receive the insulin sample; or
(ii) contact and extract the insulin sample.

10. The tester device of claim 1, further comprising a wicking pad that couples to the test pad.

11. The tester device of claim 1, wherein the label can be detected when the insulin sample comprises at least 10% amyloid fibrils.

12. A test kit, comprising:
a tester comprising:
a tester housing;
a tester substrate located in the tester housing, the tester substrate comprising:
a conjugate pad that receives an insulin sample, wherein the conjugate pad includes a label-conjugated anti-amyloid fibrils antibody; and
a test pad that couples to the conjugate pad to receive the label-conjugated anti-amyloid fibrils antibody, the test pad comprising a region treated with a second anti-amyloid fibrils antibody, wherein the second anti-amyloid fibrils antibody binds the amyloid fibrils, wherein the amyloid fibrils are also bound to the label-conjugated anti-amyloid fibrils antibody, thereby generating a visual indication of presence of amyloid fibril in the insulin sample.

13. The test kit of claim 12, wherein the nanoparticle-conjugated anti-amyloid fibrils antibody has a concentration of from about 1 mg/mL to about 4 mg/mL.

14. The test kit of claim 12, wherein the test pad includes:
(i) an indication line or stripe of anti-amyloid fibrils antibody configured to bind to amyloid fibrils-conjugated antibody complexes; and
(ii) a second indication line or stripe as the control line configured to bind to unbound conjugated antibody.

15. The test kit of claim 12, further comprising a buffer for nanoparticle-conjugated anti-amyloid fibrils antibody release.

16. The test kit of claim 12, wherein the tester device includes a sample pad as the tester input, the sample pad configured to:
(i) receive the insulin sample; or
(ii) contact and extract the insulin sample.

17. A method comprising:
providing a portable tester device comprising:
(i) a conjugate pad that includes label-conjugated anti-amyloid fibrils antibody; and
(ii) a test pad treated with a second anti-amyloid fibrils antibody, wherein the second anti-amyloid fibrils antibody binds the amyloid fibrils, wherein the amyloid fibrils are also bound to the label-conjugated anti-amyloid fibrils antibody, thereby generating a visual indication of the presence of amyloid fibril in the insulin sample;
receiving an insulin sample at an input of the portable tester device;
activating the label-conjugated anti-amyloid fibrils antibody with a label buffer solution; and
generating a visual indication of presence of amyloid fibril or denatured insulin protein in a conjugated insulin sample as an indication of insulin spoilage or degradation.

18. The method of claim 17, further comprising, before receiving the insulin sample at the input of the portable tester device, treating the insulin sample with a label buffer solution for label-conjugated anti-amyloid fibrils antibody release.

19. The method of claim 17, further comprising:
determining whether the insulin sample is safe for use by determining whether the sample has a pre-defined exceeded threshold amount of amyloid fibrils, wherein presence of exceeded threshold amount of amyloid fibrils indicates the insulin sample is not safe for use.

20. The method of claim 17, further comprising:
determining whether the insulin sample has amyloid fibrils, wherein if it does, a subject is treated with a different insulin sample, and if it doesn't, said subject is treated with the insulin sample which has been measured.
